(19) United States
(12) Patent Application Publication
(10) Pub. No.: US 2012/0022418 A1
(43) Pub. Date: Jan. 26, 2012

(54) SUPPORT / COMPRESSION VEST

(75) Inventors: Ricardo Gamboa, Monterey Park, CA (US); Gregory G. Cabrera, Monterey Park, CA (US)

(73) Assignee: BORG UNLIMITED INC., Monterey Park, CA (US)

(21) Appl. No.: 12/843,323

(22) Filed: Jul. 26, 2010

(51) Int. Cl. A61F 5/00; A41D 1/04

(52) U.S. Cl. 602/19; 2/102

(57) ABSTRACT

A back support and compression vest includes a garment made of a compression-like material with a fabric panel arranged about the abdomen designed to receive an adjustable back support belt. The fabric panel includes a right and left fabric panel cooperating to form a slot opening about the lower middle back and spaced generally about the middle front abdomen of wearer. The fabric panel is sufficiently wide to allow the belt to be properly positioned. The garment includes a pair of shoulder pad holders for receiving removable shoulder pads. The shoulder pads are sufficiently shaped to cover the wearer's shoulder while also extending rearward across a portion of the wearer's back and forward across a portion of the wearer's chest. The shoulder pad holders include a fabric envelope having an opening arranged generally on the inside portion of the garment along the arm aperture.

### BACKGROUND OF THE INVENTION

The present invention is generally directed to a back support. More particularly, the present invention is directed to a back support and compression vest having an integral fabric panel designed for receiving an adjustable back support belt.

Lower back disorders and the pain associated therewith have become common in today's society. Such disorders are typically caused by a combination of poor posture, faulty body mechanics, stressful living, loss of flexibility and a general decline in physical health and fitness. Lower back disorders are even more common for those occupations which put excessive and repetitive stress on the lower back. Occupations such as delivery personnel, construction workers, and the like are constantly bending and lifting various objects which can cause a new injury or aggravate an old one. There are several methods for treating such an injury. In general, the best non-surgical management techniques for such disorders include proper strengthening exercises, stretching exercises, treatment and rest. An unfortunate some have had to undergo operations to fuse vertebrae together to relieve the constant pain associated with an incurable injury. This ultimately reduces flexibility and movement of the individual. Prevention of the injury is obviously the ultimate goal and preferred method of treating back disorders.

Various back braces have been devised to help prevent injury or reinjury to the lower back by creating an outside support structure wrapping generally around the waist of a user. The back brace is usually substantially wide in width and can be pulled tight creating support and then fastened at the front. The internal strength of the support belt itself combined with the tight fit provides additional support to the user's spine thereby relieving stress. Support belts are commonly used in weight lifting due to the high stress loads and likelihood of injury. There are many weight lifting belts on the market that are designed specifically for use during lower leg lifts and squatting exercises. These belts are particularly thick and cumbersome and not intended to be worn for an extended period of time let alone while on the job for a full work day. Weight lifting belts need constant retensioning and adjustment as the weight lifter performs various movements and exercises, which is acceptable during short duration exercises. However, a weight lifting belt is too uncomfortable and would need to be readjusted too often to be adopted as a device used to prevent an occupation related back injury.

Compression shirts are a relatively new devise which add support to a person's upper torso. A compression shirt is a tight-fitting shirt made of a resiliently flexible material such as spandex. They are typically very breathable and are usually worn during exercising or during various sporting activities. They are typically excellent moisture managing garments and can help wick moisture away from the body more efficiently, keeping the user cooler and drier during exercise. Compression shirts cut down on excess muscle vibration which can cause extra fatigue as well as prevent chafing. Compression shirts can also keep the body slightly warmer to prevent injury to the muscles and joints. Some compression shirts are also made for cold weather and keep the body extra warm during play. Examples of compression shirt types are: Nike Pro Dri-fit, adidas Climalite, Under Armour HeatGear, CCC Armourfit Cold, and Nike Pro Basic Winter. Compression shirts also add additional support to the upper torso and tend to improve the posture of the user. Compression shirts can be worn underneath other garments and are usually undetectable to the outside viewer. However, compression shirts are not suitable replacements for a traditional back brace described herein.

In addition to risks of back injury, many occupations also risk injury from carrying various loads on their shoulders or when attempting to move and transport various articles which are stored above one's head. For instance, many warehouse workers have to repetitively store and retrieve packages and boxes which are stored at some height off the ground on various racks and storage bins. These items may be laid to rest on a shoulder when a worker transports it from one location to another. Also, when attempting to retrieve a package from a high location, the package may come down in a quick manner and contact a portion of the worker's body resulting in an impact load. The edge of a box may slam into the worker's chest or shoulder causing bruising. Furthermore, the corner of a box may poke into the chest and shoulder and create a pressure point where bruising is even more pronounced in a localized area. Compression shirts and support belts offer no protection in such instances.

Accordingly there is a need for a back support device that may be worn comfortably throughout the day while providing sufficient support to one's back and provide cushioning protection to one's shoulders. The present invention fulfills these needs and provides other related advantages.

### SUMMARY OF THE INVENTION

The back support and compression vest of the present invention includes a garment made of a compression-like material sufficiently resilient and flexible with a fabric panel located around the abdomen of a wearer. The fabric panel is designed to receive an adjustable back support belt which then forms an integral part of the garment. The fabric panel includes a right side fabric panel and a left side fabric panel coming together at the middle of the lower back forming a slot opening. The slot opening allows the belt to be easily placed within. The right and left side fabric panels are spaced about the middle front abdomen of the wearer, where the spacing accommodates the fastening of the belt. The belt may be a variety of support belts on the market today. The belt may be fastened with a hook and loop closure, use a buckle method, or any suitable closure technique. The fabric panel is substantially wide to allow the belt to be properly positioned in the optimal location. The garment helps to hold the belt in place while the wearer performs various tasks and movements.

The garment includes a pair of shoulder pad holders for receiving shoulder pads. The shoulder pads and holders are sufficiently shaped to cover the wearer's shoulder while also extending rearward across a portion of the wearer's back and also forward across a portion of the wearer's chest. The shoulder pads are removable so that the garment can be laundered. The shoulder pad holders each include a fabric envelope having an opening along the inside of the garment along the outer edge of the arm aperture. The opening is closed with a suitable closure technique such as a hook and loop fastener. The shoulder pads are made of a load absorbing material and are used to prevent impact injury from a wearer resting objects on one's body or from an object striking the wearer when one attempts to grab and retrieve an overhead object.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a perspective view of an exemplary support garment embodying the present invention;

FIG. 2 is a perspective view of an exemplary belt of FIG. 1;

FIG. 3 is a rear perspective view of FIG. 1 with the belt removed;

FIG. 4 is a view similar to FIG. 1 with the belt unfastened;

FIG. 5 is a rear view similar to FIG. 1;

FIG. 6 is a side view similar to FIG. 1;

FIG. 7 is a view similar to FIG. 1 with the shoulder pads removed and without the belt;

FIG. 8 is a partially enlarged view taken along line 8-8; and

FIG. 9 is a sectional view of taken along line 9-9.

### DETAILED DESCRIPTION OF THE PREFERRED EMBODIMENTS

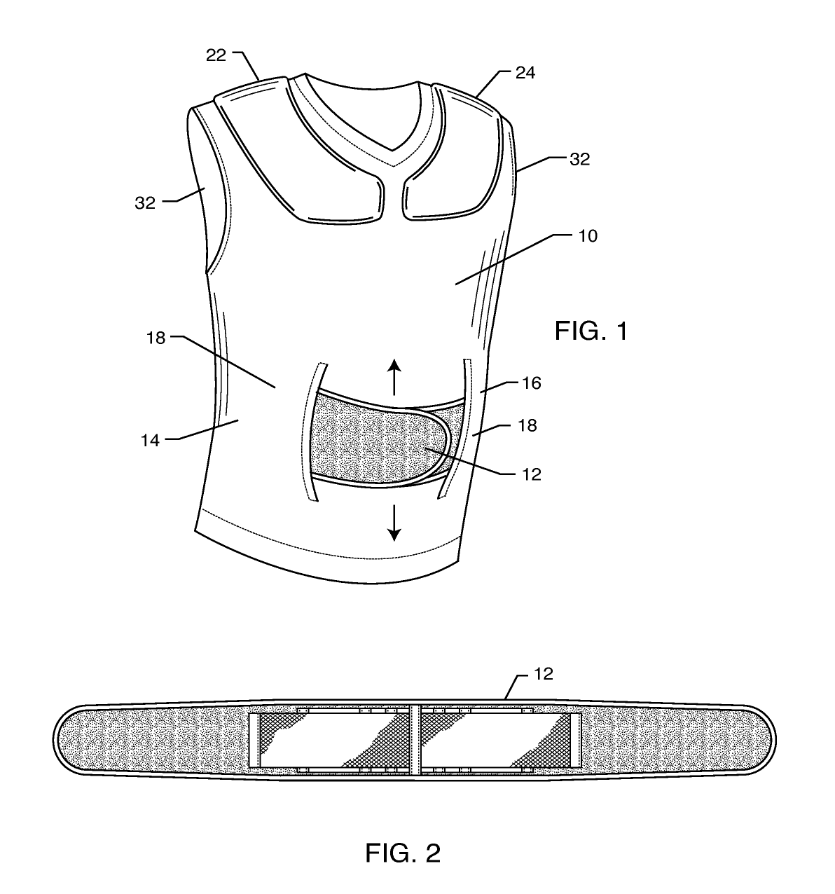

FIG. 1 is a perspective view of an exemplary garment 10 embodying the present invention. The belt 12 of FIG. 2 is placed within a right side fabric panel 14 and left side fabric panel 16 which are located around the abdomen portion of the garment 10. The fabric of the garment 10 is made of a compression-like material sufficiently resilient and flexible. Shirts made of such materials are generally referred to as compression shirts. Compression shirts are a relatively new devise which add support to a person's upper torso. A compression shirt is a tight-fitting shirt made of a resiliently flexible material such as spandex. They are typically very breathable and are usually worn during exercising or during various sporting activities. They are typically excellent moisture managing garments and can help wick moisture away from the body more efficiently, keeping the user cooler and drier during exercise. Compression shirts cut down on excess muscle vibration which can cause extra fatigue as well as prevent chafing. Compression shirts can also keep the body slightly warmer to prevent injury to the muscles and joints. Some compression shirts are also made for cold weather and keep the body extra warm during play. Compression shirts also add additional support to the upper torso and tend to improve the posture of the user. Compression shirts can be worn underneath other garments and are usually undetectable to the outside viewer.

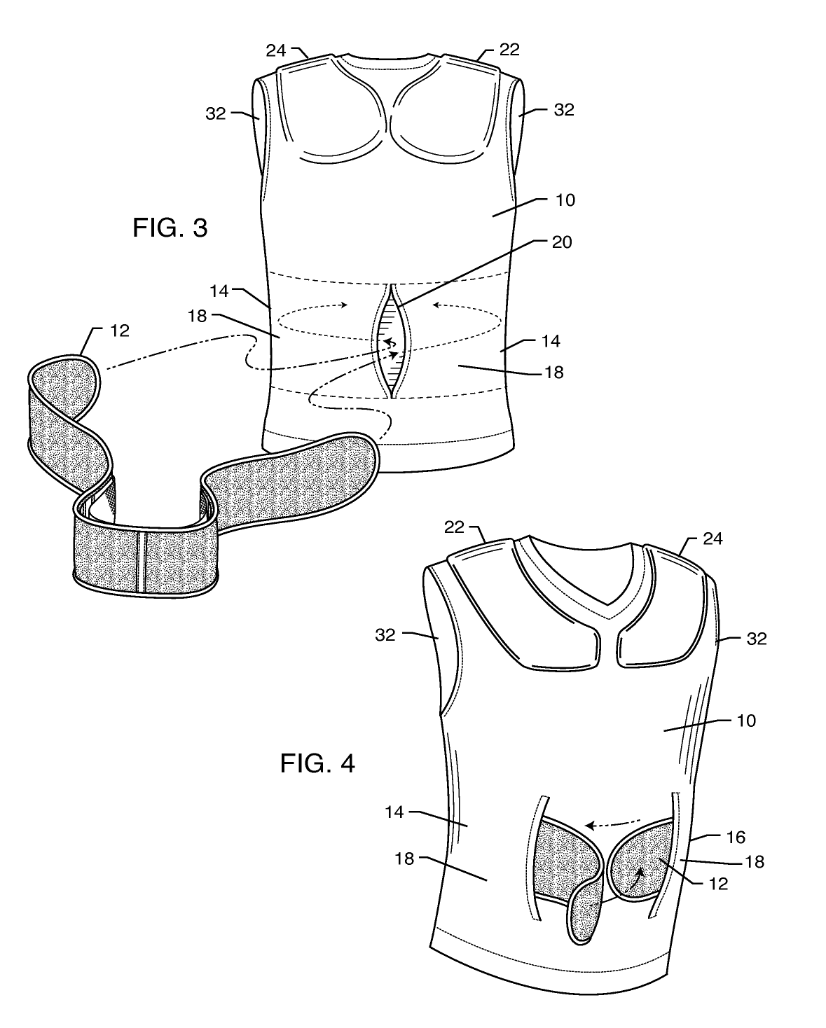

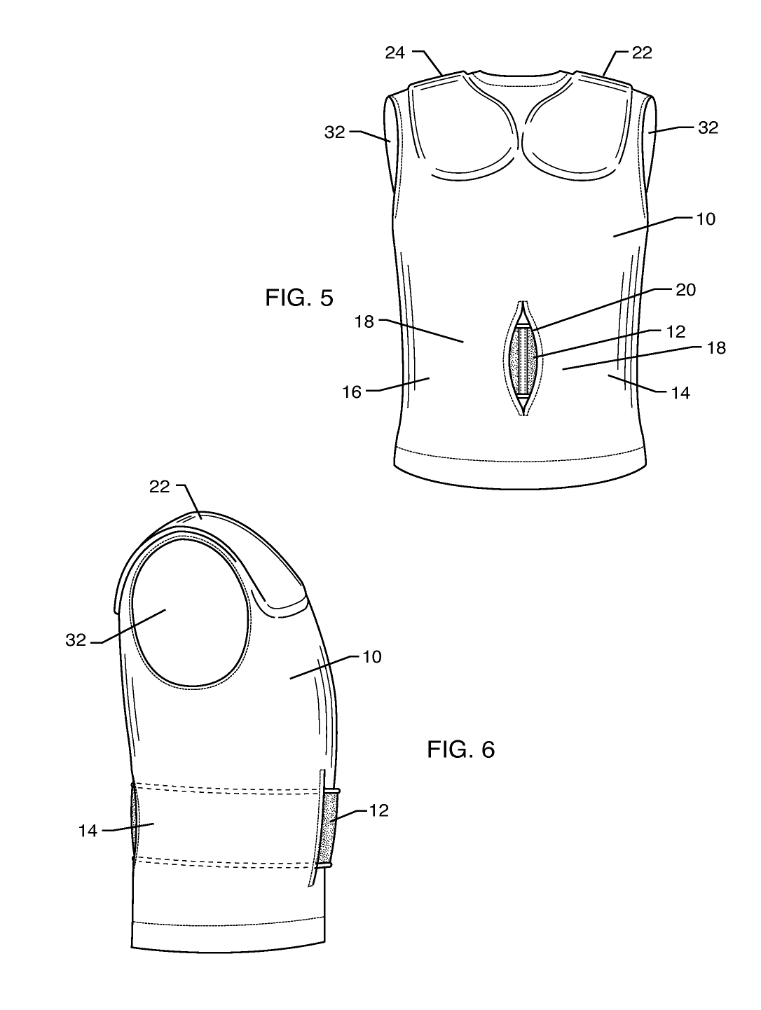

The right side fabric panel 14 and left side fabric panel 16 cooperate to form a single fabric panel 18 sufficient to receive the belt 12 such that the belt 12 forms an integral part of the garment 10. The fabric panel 18 is also made of the compression material and helps to hold and secure the belt 12 when worn. As in FIG. 3, the right side fabric panel 14 and left side fabric panel 16 cooperate to form a slot opening 20 along the middle of the lower back of the garment 10. The belt 12 is insertable and removable from the opening 20. As illustrated in FIG. 3, one end of the belt 12 can be inserted into the right side fabric panel 14 and then the other end of the belt 12 can be inserted into the left side fabric panel 16. The opening 20 facilitates the ease of inserting and removing the belt 12. Without such an opening, inserting and removing the belt 12 from the garment 10 could become overly burdensome. The belt 12 is shown as an integral part of the garment 10 in FIGS. 5 and 6.

As shown in FIG. 4, the belt 12 is fastenable at the front of the garment 10. The right side fabric panel 14 and left side fabric panel 16 are sufficiently spaced apart from each other along the front to allow the fastening and tightening of the belt 12. After the belt 12 is tightened, excess belt may be tucked within the fabric panel 18. The fabric panel 18 is designed to accommodate a range of belts 12 on the market today. The belt 12 may be a buckle type closure, a hook and loop type closure such as Velcro, or any other variation on the market today. This disclosure is not intended to limit the embodiment to any one particular type of belt 12. The fabric panel 18 is sufficiently wide to not only accommodate a range of belts 12, but to also allow the belt 12 to slide up or down for proper positioning. This is important because many workers wearing the belt 12 will come in varying body types. The garment 10 is designed to accommodate both thin people and heavier set people who will have to wear the belt 12 sufficiently lower and generally underneath their belly. The garment 10 extends below the average waistline to accommodate such varying body types and proper positioning. Accordingly, this means the both the right side fabric panel 14 and left side fabric panel 14 are sufficiently wide along with the slot opening 20.

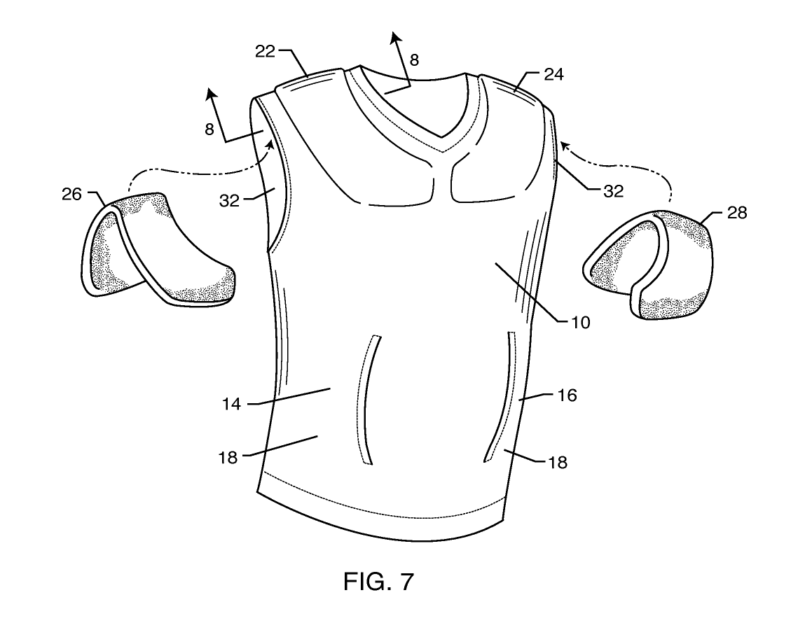

As shown in FIGS. 1-6 and more clearly in the exploded view of FIG. 7, the garment 10 includes a right shoulder pad holder 22 and left shoulder pad holder 24 for receiving a right shoulder pad 26 and a left shoulder pad 28, respectively. In addition to risks of back injury, many occupations also risk injury from carrying various loads on their shoulders or when attempting to move and transport various articles which are stored above one's head. For instance, many warehouse workers have to repetitively store and retrieve packages and boxes which are stored at some height off the ground on various racks and storage bins. These items may be laid to rest on a shoulder when a worker transports it from one location to another. Also, when attempting to retrieve a package from a high location, the package may come down in a quick manner and contact a portion of the worker's body resulting in an impact load. The edge of a box may slam into the worker's chest or shoulder causing bruising. Furthermore, the corner of a box may poke into the chest and shoulder and create a pressure point where bruising is even more pronounced in a localized area. Compression shirts and support belts offer no protection in such instances. Other occupations such a cameramen support a significant amount of weight on their shoulders for extended periods of time. Therefore, garment 10 can include protective foam support in the shoulder region.

The shoulder pads and corresponding holders are sufficiently shaped to cover the wearer's shoulder while extending rearward across a portion of the wearer's back and also forward across a portion of the wearer's chest. The shoulder pads 26 and 28 are also shaped to contour the human form so not to rub on one's neck when turning their head from side to side or become jammed when raising one's arm above their head. The material of the shoulder pads 26 and 28 are sufficiently energy absorbing and resilient to help mitigate and prevent injury from packages impacting the user's body. The compression shirt qualities of the garment 10 help hold the pads 26 and 28 in proper placement along the user's upper body. The pads 26 and 28 are removable so that the garment 10 can be laundered.

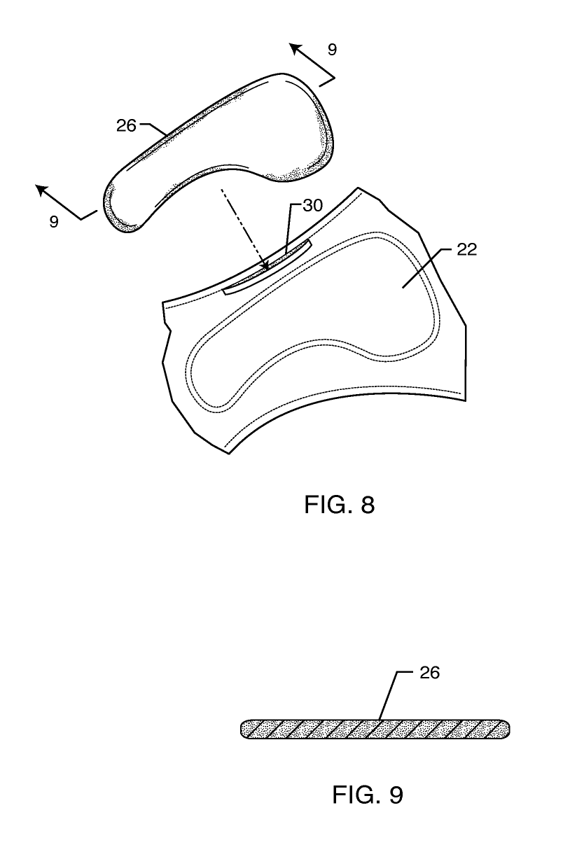

The pads 26 and 28 are insertable into the fabric envelope of the holders 22, 24 through an opening 30 as shown in FIG. 8. The opening 30 is located along the outer edge of the arm aperture 32 on the inside of the garment 10. The opening 30 is on the inside of the garment 10 so that the opening 30 never comes open and then interferes with a package or tool resting upon the wearer's shoulder. The opening 30 can be closed with any suitable method such as a hook and loop type closure such as Velcro, or any other suitable method available such as buttons, zippers, ties, and the like.

FIG. 9 shows a sectional view of the material of the right shoulder pad 26. The pad material may be made from a variety of foams and structures available today and this disclosure is not intended to limit it to any one type. The pad material is sufficiently protective to reduce the injury and stress placed upon the wearer's body when moving packages high above or when using the shoulder to rest an object thereupon. The pad material can be comprised of a multitude of impact resistance foams, or even have a hard plastic outer coating with foam attached on the other side. A wearer can also choose different levels of foam depending on the level of protection desired.

The garment 10 in this embodiment is sleeveless to aid in the removal and insertion of the shoulder pads 26 and 28. However, other sleeved variations are possible if desired such as full sleeves, half sleeves, or three-quarter sleeves. The shoulder pads 26 and 28 are also sufficiently discreet such that one can wear the garment 10 underneath normal clothing and it would be hard to the untrained observer to notice one is wearing the garment 10. This may be important for certain occupations where discretion is important, such as cameramen at formal events where they have to perform their job function while in a formal suit or tuxedo. Delivery men can wear the garment 10 underneath their normal work attire and construction workers can wear the garment 10 along with their tool belts and equipment.

Although a preferred embodiment has been described in detail for purposes of illustration, various modifications may be made without departing from the scope and spirit of the invention. Accordingly, the invention is not to be limited, except as by the appended claims.

What is claimed is:

1. A back support and compression vest, comprising:
an upper torso-receiving garment having apertures for arms and a neck of a wearer, and including adjacent fabric panels arranged generally about the abdomen of the wearer; and
an adjustable back support belt disposed within the adjacent fabric panels.

2. The vest of claim 1, wherein the garment comprises resiliently flexible material.

3. The vest of claim 1, wherein the belt is removable from the garment.

4. The vest of claim 1, wherein the belt includes a hook and loop closure.

5. The vest of claim 1, wherein the fabric panels are sufficiently wider than the belt, such that the belt may be properly positioned relative to the abdomen of the wearer.

6. The vest of claim 1, wherein the fabric panel comprises right and left fabric panels which cooperate to form a slot opening generally about the lower middle back of the wearer.

7. The vest of claim 1, wherein the garment comprises a pair of shoulder pad holders.

8. The vest of claim 1, including removable shoulder pads.

9. The vest of claim 8, wherein the shoulder pads comprise a load absorbing material.

10. The vest of claim 7, wherein the shoulder pad holders each comprise a fabric envelope having an opening arranged generally along the inside of the garment along the arm aperture.

11. The vest of claim 10, wherein each opening comprises a hook and loop closure.

12. A back support and compression vest, comprising:
an upper torso-receiving garment having apertures for arms and a neck of a wearer, and including adjacent fabric panels arranged generally about the abdomen of the wearer, wherein the fabric panels comprise right and left fabric panels which are spaced generally about the middle front abdomen of the wearer; and
an adjustable back support belt disposed within the adjacent fabric panels comprising a hook and loop closure.

13. The vest of claim 12, wherein the garment comprises resiliently flexible material.

14. The vest of claim 12, wherein the belt is removable from the garment and wherein the fabric panels are sufficiently wider than the belt, such that the belt may be properly positioned relative to the abdomen of the wearer.

15. The vest of claim 12, wherein the right and left fabric panels cooperate to form a slot opening generally about the lower middle back of the wearer.

16. The vest of claim 12, wherein the garment comprises a pair of shoulder pad holders including removable shoulder pads, wherein the shoulder pads comprise a load absorbing material.

17. The vest of claim 16, wherein the shoulder pad holders each comprise a fabric envelope having an opening arranged generally along the inside of the garment along the arm aperture.

18. The vest of claim 17, wherein each opening comprises a hook and loop closure.

19. A back support and compression vest, comprising:
an upper torso-receiving garment having apertures for arms and a neck of a wearer wherein the garment comprises resiliently flexible material, and including adjacent fabric panels arranged generally about the abdomen of the wearer wherein the fabric panels comprise right and left fabric panels which cooperate to form a slot opening generally about the lower middle back of the wearer and are spaced generally about the middle front abdomen of the wearer;
an adjustable back support belt disposed within the adjacent fabric panels wherein the belt is removable and includes a hook and loop closure, wherein the fabric panel is sufficiently wider than the belt; and
a pair of shoulder pad holders including removable shoulder pads comprising a load absorbing material, wherein the shoulder pad holders each comprise a fabric envelope having an opening arranged generally along the inside of the garment along the arm aperture wherein each opening comprises a hook and loop closure.
